SEQUENCE LISTING (2 sequences)

SEQ ID NO: 1 - protein, 4 aa, Artificial Sequence
  Synthetic peptide
  MOD_RES (1)..(1): pyroglutamic acid (pGlu)
  MOD_RES (4)..(4): C8 alkyl attached to the epsilon amine of the Lysine side chain to form Lys(Octanoyl)
ENWK

SEQ ID NO: 2 - protein, 4 aa, Artificial Sequence
  Synthetic peptide
  MOD_RES (1)..(1): pyroglutamic acid (pGlu)
ENWT

(19) United States
(12) Patent Application Publication
(10) Pub. No.: US 2024/0252581 A1
(43) Pub. Date: Aug. 1, 2024

(54) COMPOSITIONS AND METHODS FOR TREATING RESPIRATORY DISTRESS

(71) Applicant: S.I.S. SHULOV INNOVATIVE SCIENCE LTD., Rehovot (IL)

(72) Inventor: Naftali PRIMOR, Jerusalem (IL)

(73) Assignee: S.I.S. SHULOV INNOVATIVE SCIENCE LTD., Rehovot (IL)

(21) Appl. No.: 18/565,228

(22) PCT Filed: May 30, 2022
(86) PCT No.: PCT/IL2022/050573

§ 371 Date: Nov. 29, 2023

Related U.S. Application Data
(60) Provisional application No. 63/195,071, filed on May 31, 2021.

(51) Int. Cl. A61K 38/07; A61K 9/00; A61K 45/06; A61P 11/00; A61P 29/00

(52) U.S. Cl. CPC A61K 38/07; A61K 9/0078; A61K 45/06; A61P 11/00; A61P 29/00

(57) ABSTRACT

The present invention relates to the treatment or prevention of respiratory distress using a pharmaceutical composition containing a peptide denoted ZEP3, ZEP4 or a salt thereof.

### FIELD OF THE INVENTION

The present invention relates to a method of treating a subject suffering from respiratory distress comprising administering to the subject a pharmaceutical composition comprising a peptide denoted ZEP3 or ZEP4 and pharmaceutically acceptable salts thereof.

### BACKGROUND OF THE INVENTION

Respiratory distress is a condition that occurs when oxygenation and/or ventilation are insufficient thereby resulting in reduced arterial partial pressure of oxygen (PaO₂) and arterial oxygen saturation (SpO₂). When SpO₂ values are lower than 90%, oxygen supplementation is typically required. At even lower SpO₂ values, mechanical breathing support is used.

Respiratory distress may be caused by a variety of conditions. In premature infants, it is caused by deficiency of surfactant, the phospholipid mixture that reduces alveolar surface tension and decreases the pressure needed to keep the alveoli inflated and maintain alveolar stability. In young children, it may be caused by disorders of the extrathoracic or intrathoracic airways, alveoli, pulmonary vasculature, pleural spaces, or thorax.

Respiratory distress may also be secondary to respiratory, cardiovascular, hematologic, or central nervous system diseases. Diabetic patients that develop ketoacidosis are at high risk of developing respiratory distress due to hyperpnea. At high and medium altitude environments (1,500-3,500 meters altitude), illness syndromes that result from hypoxia can occur thereby leading to high-altitude pulmonary edema (HAPE) and consequently low PaO₂ and SpO₂ values.

Acute respiratory distress syndrome (ARDS) typically occurs in adults whereby a buildup of fluids in the alveoli of the lungs deprives oxygen from the bloodstream and organs. It may be caused by trauma or illness affecting the lungs.

Coronaviruses (CoVs) are a large family of viruses belonging to the Coronavirinae subfamily. There are three coronaviruses that are known to cause serious life-threatening infections in people, namely Severe Acute Respiratory Syndrome (SARS)-CoV, Middle East Respiratory Syndrome (MERS)-CoV, and SARS-COV-2, the latter was identified as the cause of a pandemic of viral pneumonia disease named COVID-19.

The most commonly observed symptoms of COVID-19 infection are fever, dry cough, and dyspnea. CT scans of the lungs of patients experiencing dyspnea and tachypnea reveal unilateral or bilateral ground-glass opacities that might progress to more clear consolidations throughout the disease. These opacities are accompanied by an increase in plasma concentrations of D-dimers which is typical for angioedema (van de Veerdonk et al. Kinins and cytokines in COVID-19: A comprehensive pathophysiological approach, doi: 10.20944/preprints202004.0023.v1).

Some COVID-19 patients develop severe ARDS with high mortality. This high severity is dependent on a cytokine storm, most likely induced by the interleukin-6 (IL-6) amplifier, which is hyper-activation machinery that regulates the nuclear factor kappa B (NF-κB) pathway and is stimulated by the simultaneous activation of IL-6-signal transducer and activator of transcription 3 (STAT3) and NF-κB signaling in non-immune cells including alveolar epithelial cells and endothelial cells (Hojyo et al. Inflamm. Regen. 2020; 40: 37. doi: 10.1186/s41232-020-00146-3).

Tolourian et al. (COVID-19 interactions with angiotensin-converting enzyme 2 (ACE2) and the kinin system; looking at a potential treatment, J. Renal Inj. Prev. 2020; 9(2): e19) disclosed that angiotensin-converting enzyme-2 (ACE2) plays a key role in the pathogenesis of COVID-19. In particular, SARS-COV-2 uses ACE2 as the receptor to enter the host cell. Binding to ACE2 was shown to occur via the spike glycoprotein expressed on the viral envelope for entering the host cells. Blocking ACE2 prevents the virus from entering the cell and is therefore desirable in order to treat COVID-19 patients.

U.S. Pat. No. 4,619,916 describes thirteen tripeptides made from L-amino acids corresponding to the formula p-GLU-X-TRP where X is a specific amino acid different from p-GLU and Trp, as well as a process for their preparation, pharmaceutical formulations containing them and use thereof as hypotensive and analgesic agents.

U.S. Pat. No. 7,220,725 and WO 2002/012269 describe novel peptides including pGlu-Asn-Trp-Lys(Octanoyl)-OH (ZEP3) and pGlu-Asn-Trp-Thr-OH (ZEP4) and pharmaceutical compositions comprising an analgesic effective amount of a peptide for topical administration in the treatment of pain.

U.S. Pat. No. 9,012,397 and WO 2012/131676 describe topical pharmaceutical compositions including the peptides ZEP3 or ZEP4 and use thereof for treating a skin disorder selected from the group consisting of Herpes viral infection, Varicella viral infection, rash, insect bites, jellyfish stings, burns, psoriasis, itching, skin allergic response, skin lesions as a result of drug or medical treatment side effects or complications, and hypopigmentation.

Gaynes et al. (Invest. Ophthalmol. Vis. Sci. 54, E-Abstract 5416, 2013) describe an analgesic effect of the peptide ZEP4 in reducing ocular pain and modifying pathways of nociception in a rat model of experimentally induced chemical corneal injury.

WO 2019/186561 describes pharmaceutical compositions comprising specific tetrapeptides for use in reducing the release or inhibiting the activity of inflammatory cytokines and mediators. The application further relates to the treatment and amelioration of symptoms associated with the release of inflammatory cytokines in inflammatory conditions including inflammatory eye disorders.

WO 2021/059266 describes compositions comprising specific tetrapeptides, for use in treating, preventing, minimizing, diminishing or reversing various signs of aging of the skin. The compositions are useful in improving the firmness or elasticity of skin, smoothing of fine lines or wrinkles, reducing skin pores and hyperpigmentation, and increasing skin thickness, radiance and/or softness.

WO 2021/059267 describes pharmaceutical compositions comprising specific tetrapeptides, for use in treating, preventing, minimizing, diminishing or reversing degenerative, age-related and trauma-induced disorders, particularly of the eye.

There remains an unmet need for compositions and methods of treating or preventing respiratory distress.

### SUMMARY OF THE INVENTION

The present invention provides compositions useful in treating or preventing respiratory distress, the compositions comprising a peptide denoted as ZEP3 or ZEP4, or pharmaceutically acceptable salts thereof. The present invention further provides methods of use of the compositions for increasing a decreased level of blood oxygen saturation or decreasing an increased level of respiratory rate of subjects suffering from respiratory distress. Further provided within the scope of the present invention is the treatment of subjects infected with coronavirus or having COVID-19, particularly those suffering from acute respiratory distress syndrome (ARDS), comprising the administration of a pharmaceutical composition comprising ZEP3 or ZEP4, or pharmaceutically acceptable salts thereof.

It is now disclosed for the first time that pharmaceutical compositions comprising ZEP3, ZEP4 or salts thereof are useful in treating respiratory distress by increasing blood oxygen saturation levels. Thus, the compositions disclosed herein are effective in treating subjects experiencing reduced blood oxygen saturation levels thereby restoring normal arterial partial pressure of oxygen (PaO₂) and arterial oxygen saturation (SpO₂) values. Also disclosed herein for the first time is the use of a pharmaceutical composition comprising ZEP3, ZEP4 or salts thereof in treating a patient infected with SARS-COV-2 and reducing or ameliorating the symptoms experienced by COVID-19 patients. The present invention is based, in part, on the unexpected findings that a pharmaceutical composition comprising the sodium salt of the peptide denoted ZEP3 tested in an acute inflamed lung model was effective in significantly increasing oxygen saturation levels, inhibiting the presence of neutrophils in the lungs, and reducing ACE2 mRNA expression. ZEP3 sodium also significantly reduced certain lung's inflammatory cytokines thereby being effective in preventing the cytokine storm which often occurs in severe COVID-19 patients.

According to a first aspect, there is provided a composition comprising a therapeutically effective amount of a peptide selected from pGlu-Asn-Trp-Lys(Octanoyl)-OH (SEQ ID NO: 1), pGlu-Asn-Trp-Thr-OH (SEQ ID NO: 2), and pharmaceutically acceptable salts thereof for use in treating or preventing respiratory distress in a subject in need thereof. Thus, according to one embodiment, the present invention provides a method of treating or preventing respiratory distress in a subject in need thereof, the method comprising administering to the subject a pharmaceutical composition comprising a therapeutically effective amount of a peptide selected from pGlu-Asn-Trp-Lys(Octanoyl)-OH (SEQ ID NO: 1), pGlu-Asn-Trp-Thr-OH (SEQ ID NO: 2), and pharmaceutically acceptable salts thereof. Each possibility represents a separate embodiment. According to another embodiment, the present invention provides the use of a peptide selected from pGlu-Asn-Trp-Lys(Octanoyl)-OH (SEQ ID NO: 1), pGlu-Asn-Trp-Thr-OH (SEQ ID NO: 2), and pharmaceutically acceptable salts thereof for the preparation of a medicament for treating or preventing respiratory distress in a subject in need thereof.

According to some embodiments, the subject is afflicted with a disease or disorder selected from pulmonary hypertension, acute chest syndrome, infectious lung disease, hypoxemia, respiratory failure, respiratory distress syndrome, acute respiratory distress syndrome, acute lung injury, pulmonary embolism, sleep apnea, altitude illness, diabetic ketoacidosis, and respiratory distress caused by lung cancer. Each possibility represents a separate embodiment. According to other embodiments, the subject is afflicted with a disease or disorder selected from pulmonary hypertension, acute chest syndrome, hypoxemia, respiratory failure, respiratory distress syndrome, acute respiratory distress syndrome, acute lung injury, pulmonary embolism, sleep apnea, altitude illness, diabetic ketoacidosis, and respiratory distress caused by lung cancer. Each possibility represents a separate embodiment. According to yet other embodiments, the subject is engaged in excessive exercise or practices excess smoking.

According to various embodiments, treating respiratory distress comprises increasing a decreased level of blood oxygen saturation in the subject. According to other embodiments, treating respiratory distress comprises decreasing an increased level of respiratory rate of the subject.

According to another aspect, there is provided a composition comprising a therapeutically effective amount of a peptide selected from pGlu-Asn-Trp-Lys(Octanoyl)-OH (SEQ ID NO: 1), pGlu-Asn-Trp-Thr-OH (SEQ ID NO: 2), and pharmaceutically acceptable salts thereof for use in treating a subject infected with coronavirus. Thus, according to certain embodiments, the present invention provides a method of treating a subject infected with coronavirus, the method comprising administering to the subject a pharmaceutical composition comprising a therapeutically effective amount of a peptide selected from pGlu-Asn-Trp-Lys(Octanoyl)-OH (SEQ ID NO: 1), pGlu-Asn-Trp-Thr-OH (SEQ ID NO: 2), and pharmaceutically acceptable salts thereof. Each possibility represents a separate embodiment. According to other embodiments, the present invention provides the use of a peptide selected from pGlu-Asn-Trp-Lys(Octanoyl)-OH (SEQ ID NO: 1), pGlu-Asn-Trp-Thr-OH (SEQ ID NO: 2), and pharmaceutically acceptable salts thereof for the preparation of a medicament for treating a subject infected with coronavirus.

According to another aspect, there is provided a composition comprising a therapeutically effective amount of a peptide selected from pGlu-Asn-Trp-Lys(Octanoyl)-OH (SEQ ID NO: 1), pGlu-Asn-Trp-Thr-OH (SEQ ID NO: 2), and pharmaceutically acceptable salts thereof for use in treating a subject having COVID-19. Thus, according to further embodiments, the present invention provides a method of treating a subject having COVID-19, the method comprising administering to the subject a pharmaceutical composition comprising a therapeutically effective amount of a peptide selected from pGlu-Asn-Trp-Lys(Octanoyl)-OH (SEQ ID NO: 1), pGlu-Asn-Trp-Thr-OH (SEQ ID NO: 2), and pharmaceutically acceptable salts thereof. Each possibility represents a separate embodiment. According to additional embodiments, the present invention provides the use of a peptide selected from pGlu-Asn-Trp-Lys(Octanoyl)-OH (SEQ ID NO: 1), pGlu-Asn-Trp-Thr-OH (SEQ ID NO: 2), and pharmaceutically acceptable salts thereof for the preparation of a medicament for treating a subject having COVID-19.

According to some embodiments, the coronavirus is β-coronavirus. According to other embodiments, the β-coronavirus is Severe Acute Respiratory Syndrome (SARS)-CoV-2. According to further embodiments, the subject is mildly infected with SARS-COV-2. According to yet other embodiments, the subject is severely infected with SARS-COV-2. According to certain embodiments, the subject infected with coronavirus suffers from a cytokine storm. According to other embodiments, the subject infected with coronavirus suffers from a respiratory syndrome. According to further embodiments, the respiratory syndrome is selected from the group consisting of lung angioedema, pulmonary embolism, viral pneumonia, severe acute respiratory syndrome, and acute respiratory distress syndrome (ARDS). Each possibility represents a separate embodiment.

According to some embodiments, the peptide has an amino acid sequence as set forth in SEQ ID NO: 1 or a salt thereof.

According to other embodiments, the peptide has an amino acid sequence as set forth in SEQ ID NO: 2 or a salt thereof.

According to various embodiments, the pharmaceutical composition comprises a sodium salt of a peptide having a sequence as set forth in any one of SEQ ID NO: 1 and SEQ ID NO: 2. Each possibility represents a separate embodiment.

According to certain embodiments, the pharmaceutical composition comprises from about 0.1% to about 5% w/w of the peptide or salt thereof, including each value within the specified range. According to particular embodiments, the pharmaceutical composition comprises from about 0.5% to about 2% w/w of the peptide or salt thereof, including each value within the specified range.

According to some embodiments, the therapeutically effective amount of the peptide or salt thereof ranges from about 0.001 mg/kg to about 1,000 mg/kg, including each value within the specified range. According to additional embodiments, the therapeutically effective amount of the peptide or salt thereof ranges from about 0.1 mg/day to about 1,000 mg/day, including each value within the specified range.

In some embodiments, the administration is intratracheal. In other embodiments, the administration is intrabronchial. In further embodiments, the administration is intranasal. In other embodiments, the administration is intra-alveolar. In yet other embodiments, the peptide or salt thereof is administered via inhalation using a nebulizer or an inhaler.

In additional embodiments, the pharmaceutical composition is in the form of a solution, a suspension, a powder or a spray. Each possibility represents a separate embodiment. In further embodiments, the pharmaceutical composition is in the form of an aerosol. In particular embodiments, the pharmaceutical composition is in the form of an aerosol comprising droplets having a mass median aerodynamic diameter (MMAD) of about 0.01 to about 100 microns, including each value within the specified range.

In certain embodiments, the pharmaceutical composition further comprises a pharmaceutically acceptable excipient. In particular embodiments, the pharmaceutically acceptable excipient comprises at least one of a binder, a filler, a diluent, a surfactant or emulsifier, a glidant or lubricant, a buffering or pH adjusting agent, a tonicity enhancing agent, a wetting agent, a thickening agent, a suspending agent, a preservative, an antioxidant, a solvent, a flavoring agent, a colorant, and a mixture or combination thereof. Each possibility represents a separate embodiment.

According to additional embodiments, the pharmaceutical composition is co-administered with at least one other active agent. According to further embodiments, the at least one other active agent is an anti-viral agent. According to other embodiments, the at least one other active agent is selected from the group consisting of chloroquine, quercetin, vitamin D, hispidulin, cirsimaritin, sulfasalazine, artemisin, curcuma, and a mixture or combination thereof. Each possibility represents a separate embodiment. According to additional embodiments, co-administration of the therapeutic agents is performed in a regimen selected from a single combined composition, separate individual compositions administered substantially at the same time, and separate individual compositions administered under separate schedules. Each possibility represents a separate embodiment.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE INVENTION

The present invention provides methods for treating a subject suffering from or is at a risk of developing respiratory distress, for example a subject infected with coronavirus, comprising administering to the subject a pharmaceutical composition comprising a peptide denoted as ZEP3, ZEP4 or salts thereof. The present invention further provides pharmaceutical compositions comprising a peptide denoted as ZEP3, ZEP4 or salts thereof for use in treating a subject suffering from or is at a risk of developing respiratory distress.

Unexpectedly, the pharmaceutical composition of the present invention provides an effective treatment of respiratory distress by elevating the arterial partial pressure of oxygen (PaO₂) and arterial oxygen saturation (SpO₂) in subjects with reduced values of PaO₂ and SpO₂. The present invention is based in part on the unexpected finding that a statistically significant increase in oxygen saturation levels was detected in an acute inflamed lung model already 12 hours after a single administration of the sodium salt of the peptide denoted ZEP3. The increase was exerted at least 48 hours after administration. The present invention further provides methods of use of the pharmaceutical composition for relieving various symptoms associated with COVID-19 thereby preventing disease deterioration. Surprisingly, a single intratracheal administration of ZEP3 sodium tested in an acute inflamed lung model was effective in inhibiting the presence of neutrophils in the lungs and was further shown to reduce ACE2 mRNA expression. ZEP3 sodium also significantly reduced lung's inflammatory cytokines including, but not limited to, IL-1a, IL-6, IL-12, IL-17, KC, MCP-1, TNF-α, CXCL10, and G-CSF which indicates its efficacy in preventing the cytokine storm which often occurs in severe COVID-19 patients.

According to certain aspects and embodiments, the pharmaceutical compositions of the present invention comprise a peptide denoted ZEP3 having the following sequence pGlu-Asn-Trp-Lys(Octanoyl)-OH (SEQ ID NO: 1), wherein pGlu is pyroglutamic acid or a pharmaceutically acceptable salt thereof. ZEP3 can be produced, for example, by the procedure described in U.S. Pat. No. 7,220,725. ZEP3 has a C₈ alkyl (herein octanoyl) attached by an amide linkage to the side chain of a Lys residue of the peptide sequence (Lys(Octanoyl)). The skilled in the art can appreciate that lysine has an amino-containing side chain. As such, peptides encompassing lysine may be modified through said lysine side chain amino functionality. Specifically, the lysine side chain amino group is a primary amine (—NH₂), which is convertible to an amide by its reaction with a carboxylic acid containing moiety. It is to be understood that the term “Lys(Octanoyl)” refers to the product of such a reaction, wherein the lysine amino side chain is reacted with octanoic acid thereby forming an octanoyl amide (C₇H₁₅C(O)NH) comprising an octanoyl group (C₇H₁₅C(O)). It is further to be understood that when referring to “C₈ alkyl” in the context of the chemical substitution of the lysine's amino side chain, the reference is to the group chemically bonded to the carbonyl. In other words, reference is made to a fragment having the chemical structure RC(O)NH, wherein R is an alkyl group which is C₇H₁₅.

According to certain aspects and embodiments, the pharmaceutical compositions of the present invention comprise a peptide denoted ZEP4 having the following sequence pGlu-Asn-Trp-Thr-OH (SEQ ID NO: 2) wherein pGlu is pyroglutamic acid or a pharmaceutically acceptable salt thereof. ZEP4 can be produced, for example, by the following procedure:

The synthesis of ZEP4 may be performed by a sequential synthesis of 9-fluoromethoxycarbonyl (Fmoc) amino acids on a solid support of chlorotrityl chloride resin (CTC). CTC resin (125 gr) is loaded with Fmoc-threonine (t-butyl; 79 gr) and diisopropyl cthylamine (DIPEA; 160 gr) is used as the coupling agent of the amino acid to the solid support. The Fmoc protecting group is removed by a mixture of 25% piperidine and dimethylformamide (DMF) and the resin-peptide is filtered and washed with DMF. A second amino acid, Fmoc-Trp (85 gr), is activated by a mixture of (2-(1H-benzotriazol-1-yl)-1,1,3,3-tetramethyluronium hexafluorophosphate (HBTU)/hydroxy benzothiazole (OHBT) coupled to the first amino acid by addition of DIPEA. The Fmoc group is removed as described hereinabove and the resin-peptide is filtered and washed with DMF. A third amino acid, Fmoc-Asn (trt) (119 gr) is activated by HBTU/HOBT and coupled by addition of DIPEA. The Fmoc group is removed as described hereinabove and the resin-peptide is filtered and washed with DMF. A fourth amino acid pGlu (26 gr) is activated by HBTU/HOBT and coupled by DIPEA.

The peptide-resin is thoroughly washed with DMF followed by IPA and dried under reduced pressure. The peptide is cleaved from the resin and protecting groups of the Thr and Asn as well by TFA (95%) and triisopropyl silane (TIS) (5%) at room temperature for 2 hours. The peptide is precipitated by addition of methyl tert-butyl ether (MTBE), filtered and dried (yield 46 gr).

The crude product (46 gr) is dissolved in a mixture of acetonitrile (ACN) and water and loaded on preparative HPLC system (4″, RP C-18 100-120 A pore size) and purified using a gradient system containing Phase A—0.1% TFA in water; and Phase B—ACN. The elution is done by gradually increasing phase B (3% to 33%) during 45 minutes. Fractions having purity greater than 97% are collected. The combined fractions are eluted on the same HPLC system using a gradient containing Phase A: 0.2% acetic acid; and Phase B: ACN. The elution is performed by gradually increasing phase B (10% to 40%) during 30 minutes. Fractions having purity greater than 97% are collected, combined, and lyophilized (yield 29 gr). The final product has M. W. (MS) of 530.5; and 97.3% purity (HPLC).

The peptides ZEP3 and ZEP4 can be incorporated into the compositions as salts. As used herein, the term “salts” refers to salts of carboxyl groups also termed base addition salts and to acid addition salts of amino or guanidino groups of the peptide molecule. Suitable base addition salts include, but are not limited to, metallic salts of sodium, calcium, lithium, magnesium, potassium, aluminum, ferric and zinc; ammonium salts derived from ammonia, primary, secondary, tertiary and quaternary amines, non-limiting examples of which are trimethylamine, cyclohexylamine, benzylamine, dibenzylamine, 2-hydroxyethylamine, bis(2-hydroxyethyl)amine, phenylethylbenzylamine, dibenzylethylenediamine, procaine, chloroprocaine, piperidine, monoethanolamine, triethanolamine, quinine, choline, and N-methylglucosamine. Each possibility represents a separate embodiment. Salts with amino acids such as glycine, ornithine, histidine, phenylglycine, lysine, and arginine are contemplated. Each possibility represents a separate embodiment. Furthermore, any zwitterionic salts formed by a carboxylic acid and amino or guanidino groups of the peptide molecule are contemplated as well.

Suitable acid addition salts include salts derived from inorganic acids such as, but not limited to, hydrochloric, nitric, phosphoric, sulfuric, hydrobromic, hydriodic, phosphorous, and the like, as well as salts derived from organic acids such as aliphatic mono- and di-carboxylic acids such as acetic acid or oxalic acid, phenyl-substituted alkanoic acids, hydroxy alkanoic acids, alkanedioic acids, aromatic acids, aliphatic and aromatic sulfonic acids and the like. Each possibility represents a separate embodiment. The salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Each possibility represents a separate embodiment. Also contemplated are salts of amino acids such as arginate and the like and gluconate or galacturonate. Each possibility represents a separate embodiment.

The acid addition salts may be prepared by known methods of the art in which the free base form is brought into contact with a sufficient amount of the desired acid to produce the salt. The base addition salts may be prepared by known methods of the art in which the free acid form is brought into contact with a sufficient amount of the desired base to produce the salt.

In one embodiment of the present invention, the peptide is the sodium salt of ZEP3 (pGlu-Asn-Trp-Lys(Octanoyl)-OH·nNa wherein n is 1 or 2; hereinafter referred to as “ZEP3 sodium salt” or “ZEP3Na”). In particular embodiments, the sodium salt of ZEP3 comprises the following formula: pGlu-Asn-Trp-Lys(Octanoyl)-ONa. ZEP3 sodium salt can be produced, for example, by the following procedure:

ZEP3 (3.1 g) is solubilized in NaHCO₃ (100 mM) in water (50 g/l). The solution is injected into an HPLC ion exchange column (2.5×22 cm Luna C18,100A, 15 micron) and eluted by a gradient consisting of: Mobile Phase A: NaHCO₃ 2 mM in H₂O; Mobile Phase B: NaHCO₃ 2 mM in CH₃CN/H₂O (8/2); and Mobile Phase C: NaHCO₃100 mM in water. Loading per run: 5% maximum (W/W % Peptide/Stationary Phase). Flow: 4.8 cm/min (24 ml/min). The gradient procedure is as follows: 20 min phase C; 5 min Phase A; 18 min Phase B; and 7 min Phase C. A fraction containing the product is collected and concentrated under reduced pressure to remove acetonitrile (110 g/l) then freeze dried [yield 2.2 g (71%)]. The final product has 99.7% purity (HPLC), 3.1% sodium content and solubility of 50 mg/ml water.

In another embodiment of the present invention, the peptide is the sodium salt of ZEP4 (pGlu-Asn-Trp-Thr-OH.nNa wherein n is 1 or 2; hereinafter referred to as “ZEP4 sodium salt” or “ZEP4Na”). In particular embodiments, the sodium salt of ZEP4 comprises the following formula: pGlu-Asn-Trp-Thr-ONa. ZEP4 sodium salt can be produced, for example, by the following procedure:

ZEP4 (5 g) is solubilized in NaHCO₃ (100 mM) in water (50 g/l). The solution is injected into an HPLC ion exchange column (2.5×22 cm Luna C18,100A, 15 micron) and eluted by a gradient consisting of: Mobile Phase A: NaHCO₃ 2 mM in H₂O; Mobile Phase B: NaHCO₃ 2 mM in CH₃CN/H₂O (8/2); and Mobile Phase C: NaHCO₃100 mM in water. Loading per run: 5% maximum (W/W % Peptide/Stationary Phase). Flow: 4.8 cm/min (24 ml/min). The gradient procedure is as follows: 20 min phase C, then 5 min Phase A, then 20 min Phase B, and 10 min Phase C. A fraction containing the product is collected and concentrated under reduced pressure to remove acetonitrile (110 g/l) then freeze dried [yield 4 g (80%)]. The final product has 97.5% purity (HPLC), 2.5% sodium content and solubility of 50 mg/ml water.

According to the principles of the present invention, the pharmaceutical composition comprises a therapeutically effective amount of ZEP3, ZEP4, or salts thereof. The term “therapeutically effective amount” as used herein refers to the amount of the active agent which is effective to abate, alleviate and/or treat respiratory distress. Typically, the therapeutically effective amount of the peptide or salt thereof ranges from about 0.001 mg/kg to about 1,000 mg/kg, including each value within the specified range. Exemplary amounts include, but are not limited to, 0.001 mg/kg. 0.05 mg/kg, 0.01 mg/kg, 0.5 mg/kg, 1 mg/kg, 5 mg/kg, 10 mg/kg, 50 mg/kg, 100 mg/kg, 500 mg/kg, or 1,000 mg/kg, with each possibility representing a separate embodiment. Additionally or alternatively, the therapeutically effective amount of ZEP3, ZEP4 or salts thereof may range from about 0.1 mg/day to about 1,000 mg/day, including each value within the specified range. Exemplary amounts include, but are not limited to, 0.1 mg/day. 0.5 mg/day, 1 mg/day, 5 mg/day, 10 mg/day, 20 mg/day, 40 mg/day, 60 mg/day, 80 mg/day, 100 mg/day, 120 mg/day. 150 mg/day, 175 mg/day, 200 mg/day, 250 mg/day, 300 mg/day, 400 mg/day, 500 mg/day, 600 mg/day, 700 mg/day, 800 mg/day, 900 mg/day, or 1,000 mg/day, with each possibility representing a separate embodiment.

The peptides of the present invention can be used as pharmaceutical agents per se or as part (active ingredient) of a pharmaceutical composition together with a pharmaceutically acceptable excipient. In accordance with these embodiments, the composition may comprise from about 0.1% to about 5% w/w of the peptide, including each value within the specified range. According to other embodiments, the composition comprises from about 0.5% to about 2% w/w of the peptide, including each value within the specified range. According to yet other embodiments, the composition comprises about 1% of the peptide. In various embodiments, the amount of peptide ranges from about 200 μg to about 800 μg per gram composition, including each value within the specified range. In further embodiments, the amount of peptide ranges from about 300 μg to about 700 μg per gram composition, including each value within the specified range. In additional embodiments, the amount of peptide ranges from about 400 μg to about 600 μg per gram composition, including each value within the specified range. In particular embodiments, the amount of peptide is about 500 μg per gram composition.

As used herein, the term a “pharmaceutical composition” refers to a preparation of the peptide or salt thereof with one or more chemical components such as pharmaceutically acceptable excipients designed to facilitate administration of the peptide to a subject, preferably a human subject. The term “pharmaceutically acceptable excipient” as used herein refers to an excipient that does not abrogate the beneficial therapeutic activity and properties of the peptide of the present invention. Suitable pharmaceutically acceptable excipients within the scope of the present invention include, but are not limited to, a binder, a filler, a diluent, a surfactant or emulsifier, a glidant or lubricant, a buffering or pH adjusting agent, a tonicity enhancing agent, a wetting agent, a thickening agent, a suspending agent, a preservative, an antioxidant, a solvent, a flavoring agent, a colorant, and a mixture or combination thereof. Each possibility represents a separate embodiment.

Suitable binders include, but are not limited to, povidone (PVP: polyvinyl pyrrolidone), copovidone, hydroxypropyl cellulose (HPC), hydroxypropyl methylcellulose (HPMC), carboxy methyl cellulose (CMC), hydroxyethylcellulose, gelatin, polyethylene oxide, poly ethylene glycol (PEG), poly vinyl alcohol (PVA), acacia, chitin, chitosan, dextrin, magnesium aluminum silicate, starch, and polymethacrylates, or a mixture or combination thereof. Each possibility represents a separate embodiment. In one embodiment, the pharmaceutical composition comprises from about 0% to about 40% w/w of a binder, including each value within the specified range.

Suitable fillers include, but are not limited to, mica, talc, silicon dioxide, nylon, polyethylene, silica, polymethacrylate, kaolin, calcium carbonate, calcium phosphate, microcrystalline cellulose, various sugars and types of starch, polysugars, dextrin, cyclodextrins (e.g. β-CD, hydroxypropyl-β-CD, sulfobutylether-CD), and Teflon, or a mixture or combination thereof. Each possibility represents a separate embodiment. In one embodiment, the pharmaceutical composition comprises from about 0.5% to about 50% w/w of a filler, including each value within the specified range.

Suitable diluents include, but are not limited to, dicalcium phosphate dihydrate, sugars, lactose, trehalose, cyclodextrins, calcium phosphate, cellulose, kaolin, mannitol, sodium chloride, and dry starch, or a mixture or combination thereof. Each possibility represents a separate embodiment. In one embodiment, the pharmaceutical composition comprises from about 0.5% to about 50% w/w of a diluent, including each value within the specified range.

Suitable surfactants are cationic, anionic or zwitterionic including, but not limited to, polyoxyethylene glycol octylphenol ethers, polyoxyethylene glycol alkylphenol ethers, polyoxyethylene glycol sorbitan alkyl esters (polysorbate 60, polysorbate 80, etc.), polyethyleneglycol tocopheryl succinate, polyethoxylated castor oil derivatives (Cremophor El, Cremophor Rh40), tyloxapol, sorbitan alkyl esters, block copolymers of polyethylene glycol and polypropylene glycol (poloxamer), dioctyl sodium sulfosuccinate, perfluorooctanesulfonate, alkylbenzene sulfonates, sodium lauryl ether sulfate, ammonium laureth sulfate, ammonium lauryl sulfate, disodium laureth sulfosuccinate, lignosulfonate, sodium stearate, benzalkonium chloride, cetyl pyridinium chloride, benzethonium chloride, cetyl trimethylammonium bromide, cetyl trimethylammonium chloride, and betaines, or a mixture or combination thereof. Each possibility represents a separate embodiment. In one embodiment, the pharmaceutical composition comprises from about 0% to about 5% w/w of a surfactant, including each value within the specified range.

Suitable emulsifiers include, but are not limited to, polyethylene glycol ethers of stearic acid such as steareth-2, steareth-4, steareth-6, steareth-7, steareth-10, steareth-11, steareth-13, steareth-15, and steareth-20, glyceryl stearate, stearyl alcohol, cetyl alcohol, cetearyl alcohol, behenyl alcohol, diethanolamine, lecithin, and polyethylene glycols, or a mixture or combination thereof. Each possibility represents a separate embodiment. In one embodiment, the pharmaceutical composition comprises from about 0% to about 5% w/w of an emulsifier, including each value within the specified range.

Suitable glidant includes, but is not limited to, silicon dioxide and suitable lubricants include, but are not limited to, sodium stearyl fumarate, stearic acid, polyethylene glycol or stearates, such as magnesium stearate, or a mixture or combination thereof. Each possibility represents a separate embodiment. In one embodiment, the pharmaceutical composition comprises from about 0% to about 5% w/w of a glidant or lubricant, including each value within the specified range.

Suitable buffering or pH adjusting agents include, but are not limited to, acidic buffering or pH adjusting agents such as short chain fatty acids, citric acid, acetic acid, hydrochloric acid, sulfuric acid and fumaric acid; and basic buffering or pH adjusting agents such as tris, tricthylamine, sodium carbonate, sodium bicarbonate, sodium hydroxide, potassium hydroxide, and magnesium hydroxide, or a mixture or combination thereof. Each possibility represents a separate embodiment. Typically, the buffering or pH adjusting agents are incorporated into the composition in amounts suitable for obtaining a pH in the range of from about 3.5 to about 8.5, including each value within the specified range. In one embodiment, the buffering or pH adjusting agents are incorporated into the composition in amounts suitable for obtaining a pH in the range of from about 4 to about 7, including each value within the specified range. In one embodiment, the pharmaceutical composition comprises from about 0% to about 1% w/w of a buffering or pH adjusting agent, including each value within the specified range.

Suitable tonicity enhancing agents include, but are not limited to, ionic and non-ionic agents. For example, ionic compounds include, but are not limited to, alkali metal or alkaline earth metal halides, such as, for example, CaCl₂), KBr, KCl, LiCl, NaI, NaBr or NaCl, and boric acid, or a mixture or combination thereof. Each possibility represents a separate embodiment. Non-ionic tonicity enhancing agents are, for example, urea, glycerol, sorbitol, mannitol, propylene glycol, and dextrose, or a mixture or combination thereof. Each possibility represents a separate embodiment. In one embodiment, the pharmaceutical composition comprises from about 0% to about 5% w/w of a tonicity-enhancing agent, including each value within the specified range.

Suitable wetting agents include, but are not limited to, glycerin, starches, benzododecinium bromide (BOB), and cetrimide (Cet), or a mixture or combination thereof. Each possibility represents a separate embodiment. In one embodiment, the pharmaceutical composition comprises from about 0% to about 5% w/w of a wetting agent, including each value within the specified range.

Suitable thickening agents include, but are not limited to, fatty acids and alcohols such as stearic acid and stearyl alcohol; gums such as xanthan, carrageenan, gelatin, cellulose gum, agarose, karaya, pectin, amylopectin, and locust beans gum; various polymers such as hydroxypropyl methylcellulose (HPMC), hydroxypropyl cellulose (HPC), hydroxymethyl cellulose, hydroxyethyl cellulose, methyl cellulose, calcium carboxymethyl cellulose, polyvinylpyrrolidone (povidone, PVP), polyvinyl alcohol, medium to high molecular weight polyethylene glycols (PEG-3350, PEG-6000, etc.), glucosides, tetrasodium etidronate, polyacrylic acid, polymethacrylic acid, acrylamides copolymer, sodium acrylates copolymer, sodium alginate, calcium alginate, magnesium alginate, alginic acid, hyaluronic acid, polyglucuronic acid (poly-α- and -β-1,4-glucuronic acid), chondroitin sulfate, furcellaran, carboxymethylcellulose, polycarboxylic acids, carbomer, bentonite, chitin, chitosan, carboxymethyl chitin, and cross-linked polyacrylate materials available under the trademark Carbopol®, or a mixture or combination thereof. Each possibility represents a separate embodiment. In one embodiment, the pharmaceutical composition comprises from about 0% to about 30% w/w of a thickening agent, including each value within the specified range.

Suitable suspending agents include, but are not limited to, acacia, alginic acid, bentonite, carbomer, carboxymethylcellulose calcium, carrageenan, colloidal silicon dioxide, dextrin, gelatin, guar gum, hydroxyl ethyl cellulose, hydroxyethyl propylcellulose, hydroxyl propyl cellulose (HPC), hydroxypropyl methylcellulose, methylcellulose, maltodextrin, microcrystalline cellulose (MCC), polydextrose, polyvinyl alcohol, povidone, propylene glycol alginate, sodium alginate, sodium carboxymethylcellulose, starch, tragacanth, and xanthan gum, or a mixture or combination thereof. Each possibility represents a separate embodiment. In one embodiment, the pharmaceutical composition comprises from about 0% to about 30% w/w of a suspending agent, including each value within the specified range.

Suitable preservatives include, but are not limited to, methylparaben, propylparaben, butylparaben, ethylparaben, benzoic acid, potassium sorbate, trisodium EDTA, benzalkonium chloride, tetrasodium EDT, edetate disodium, benzophenone, 2-bromo-2-nitropane-1,3-diol, butylated hydroxytoluene, chlorhexidine digluconate, citric acid, DMDM hydantoin, formaldehyde, methylchloroisothiazolinone, methylisothiazolinone, methyldibromo glutaronitrile, sodium benzoate, phenoxyethanol, ethyl alcohol, benzyl alcohol, chlorobutanol, thimerosal, phenylmercuric nitrate, diazolidinyl urea, imidazolidinyl urea, and quaternium-15, or a mixture or combination thereof. Each possibility represents a separate embodiment. In one embodiment, the pharmaceutical composition comprises from about 0% to about 5% w/w of a preservative, including each value within the specified range.

Suitable antioxidants include, but are not limited to, ascorbic acid, ubiquinone, tocophenyl acetate, ascorbyl palmitate, edetate disodium, and sodium bisulfite, or a mixture or combination thereof. Each possibility represents a separate embodiment. In one embodiment, the pharmaceutical composition comprises from about 0% to about 10% w/w of an antioxidant, including each value within the specified range.

Suitable solvents include, but are not limited to, water, lower alcohols such as ethanol and isopropanol, propylene glycol, ammonium xylenesulfonate, and low molecular weight polyethylene glycols such as, e.g. PEG-300, PEG-1450 etc., or a mixture or combination thereof. Each possibility represents a separate embodiment. Additional solvents include, but are not limited to, oils constituting an oil phase (e.g. in emulsion compositions). Exemplary oil phases include, but are not limited to, Miglyol 810 (medium chain triglyceride), soy lecithin (e.g. phospholipon 90), cholesterol etc., or a mixture or combination thereof. Each possibility represents a separate embodiment. In one embodiment, the pharmaceutical composition comprises from about 0% to about 99.9% w/w of a solvent, including each value within the specified range.

Suitable flavoring agents include, but are not limited to, sweeteners such as sucralose, and synthetic flavor oils and flavoring aromatics, natural oils, extracts from plants, leaves, flowers, and fruits, or a mixture or combinations thereof. Each possibility represents a separate embodiment. Exemplary flavoring agents include cinnamon oils, oil of wintergreen, peppermint oils, clover oil, hay oil, anise oil, eucalyptus, vanilla, citrus oil such as lemon oil, orange oil, grape and grapefruit oil, and fruit essences including apple, peach, pear, strawberry, raspberry, cherry, plum, pineapple, and apricot, or a mixture or combination thereof. Each possibility represents a separate embodiment. In one embodiment, the pharmaceutical composition comprises from about 0% to about 5% w/w of a flavoring agent, including each value within the specified range.

Suitable colorants include, but are not limited to, alumina (dried aluminum hydroxide), annatto extract, calcium carbonate, canthaxanthin, caramel, β-carotene, cochineal extract, carmine, potassium sodium copper chlorophyllin (chlorophyllin-copper complex), dihydroxyacetone, bismuth oxychloride, synthetic iron oxide, ferric ammonium ferrocyanide, ferric ferrocyanide, chromium hydroxide green, chromium oxide greens, guanine, mica-based pearlescent pigments, pyrophyllite, disodium dityrylbiphenyl, mica, dentifrices, talc, titanium dioxide, aluminum powder, bronze powder, copper powder, and zinc oxide, or a mixture or combination thereof. Each possibility represents a separate embodiment. In one embodiment, the pharmaceutical composition comprises from about 0% to about 5% w/w of a colorant, including each value within the specified range.

The pharmaceutical compositions of the present invention may be manufactured by processes well known in the art, e.g., by means of conventional mixing, dissolving, suspending, solubilizing, complexing, granulating, levigating, emulsifying, encapsulating, entrapping, spray-drying, and lyophilizing processes, or a combination thereof. They may be formulated in a conventional manner using one or more pharmaceutically acceptable excipients as described above, which facilitate processing of the peptides and salts into preparations which can be used as medicaments. Proper formulation is dependent upon the route of administration chosen. In one embodiment, the administration is performed nasally or orally via inhalation. In other embodiments, the pharmaceutical compositions of the present invention are formulated for intratracheal, intrabronchial, intranasal or intra-alveolar administration. Each possibility represents a separate embodiment.

The pharmaceutical composition of the present invention may be formulated in any form suitable for the administration routes indicated above. Exemplary forms within the scope of the present invention include, but are not limited to a solution, a suspension, a powder, or a spray. Each possibility represents a separate embodiment.

For intranasal administration, the compositions of the invention can be formulated as a solution or suspension, or as a spray. Typically, such solutions or suspensions are isotonic relative to nasal secretions. Preferably, the solution or suspension has a pH ranging from about 6.0 to about 7.0, including each value within the specified range. For administration by nasal inhalation, the peptide or salt is conveniently delivered in the form of an aerosol spray from a pressurized pack or a nebulizer with the use of a suitable propellant, e.g., dichlorodifluoromethane, trichlorofluoromethane, dichloro-tetrafluoroethane or carbon dioxide. Each possibility represents a separate embodiment. In the case of a pressurized aerosol, the dosage unit may be determined by providing a valve to deliver a metered amount. Capsules and cartridges of, e.g., gelatin for use in a dispenser may be formulated containing a powder mix of the peptide or salt thereof and a suitable carrier such as lactose or starch. An aerosolized liquid or powder forms are traditionally intended to substantially release or deliver the active agent to the epithelium of the lungs. In some embodiments, an aerosol is administered via oral inhalation.

Pharmaceutical compositions in the form of aerosols may be characterized by the size distribution of the droplets. Droplet sizes are typically characterized by the mass median aerodynamic diameter. The term “mass median aerodynamic diameter” (MMAD) refers to the diameter of airborne particles at which 50% of particles by mass are larger and 50% of particles by mass are smaller. Suitable droplet sizes include, but are not limited to, about 0.01 to about 100 microns, including each value within the specified range. In one embodiment, the droplets have a MMAD of about 0.01 to about 50 microns, including each value within the specified range. In another embodiment, the droplets have a MMAD of about 0.1 to about 10 microns, including each value within the specified range.

For inhalation or aspiration, the compositions of the invention can be formulated as a solution or suspension as well as a powder. If desired, the pharmaceutical composition may be administered with the aid of nasal prongs, a face mask, an enclosed tent or chamber (completely or semi-sealed), an intratracheal catheter, an endotracheal tube, or a tracheostomy tube as is known in the art for achieving intratracheal, intrabronchial, or intra-alveolar administration. Each possibility represents a separate embodiment.

The pharmaceutical composition of the invention may be formulated as controlled or sustained release formulation allowing for extended release of the peptide or salt thereof over a predetermined time period. In accordance with these embodiments, the composition may further comprise a sustained release agent such as, but not limited to, hydroxypropyl methyl cellulose, acrylic acid or (meth)acrylate-based polymers, ethyl cellulose and the like. Each possibility represents a separate embodiment.

The pharmaceutical composition of the present invention may be used in combination therapy with at least one other active agent. The combination therapy, according to the principles of the present invention, includes combined therapies that are administered individually or as a single composition. When administered individually, the separate therapeutic agents may be administered at substantially the same time or under a separate regime with each possibility representing a separate embodiment. In some embodiments, the therapeutic effects achieved as a result of the combination therapy are synergistic or cooperative. The terms “synergistic”, “cooperative” and “super-additive” used herein interchangeably refer to a therapeutic effect of the peptide or salt and the other active agent which is higher than the sum of the individual therapeutic effects of each drug administered separately. In one embodiment, the at least one other active agent is an anti-viral agent such as, but not limited to, nucleoside/nucleotide reverse transcriptase inhibitors (NRTIs), non-nucleoside reverse transcriptase inhibitors (NNRTIs), protease inhibitors (PIs), fusion inhibitors, chemokine receptor antagonists, integrase inhibitors, cytokines, and lymphokines. Each possibility represents a separate embodiment. Other active agents include, but are not limited to, chloroquine, quercetin, vitamin D, hispidulin, cirsimaritin, sulfasalazine, artemisin, curcuma, and a mixture or combination thereof. Each possibility represents a separate embodiment.

According to the principles of the present invention the pharmaceutical composition comprising ZEP3, ZEP4, or pharmaceutically acceptable salts thereof is useful in treating a subject suffering from respiratory distress or a subject who is at risk of developing respiratory distress. Each possibility represents a separate embodiment. The terms “treatment” and “treating”, used herein interchangeably, refer to curing, healing, alleviating, relieving, altering, ameliorating, or improving the disease or disorder, at least one symptom of the disease or disorder, or the progression of the disease or disorder. Specifically, as used herein, treating respiratory distress refers to at least one of increasing a decreased level of blood oxygen saturation in the subject, and decreasing an increased level of respiratory rate of the subject. Each possibility represents a separate embodiment. The treatment also includes aiding recovery or reversion of a decreased level of blood oxygen saturation or an increased level of respiratory rate to normal levels of blood oxygen saturation and respiratory rate. Each possibility represents a separate embodiment. The partial pressure of oxygen in arterial blood (PaO₂) is a measurement of the oxygen content in arterial blood (typically expressed in mmHg). The ratio of oxygen bound to hemoglobin in red blood cells (SpO₂) is a measurement of oxygen saturation (typically expressed in %). Normal individuals have 95-100% oxygen saturation, and when oxygen saturation is below 90%, the patient is considered “hypoxic” thereby experiencing a respiratory distress. As a consequence of a decrease in PaO₂ and SpO₂ levels, the respiratory rate is elevated in order to compensate for the lack of oxygen in the tissues. The respiratory rate can also be elevated in individuals experiencing ketoacidosis in order to compensate for the reduction in pH of the blood. Respiratory rates are typically reported in units of breaths per minute, i.e. the number of complete breathing cycles that occur within a sixty-second period of time. Normal respiratory rates for healthy adults at rest are between 12-20 breaths per minute.

According to some aspects and embodiments, “treatment” or “treating” a subject suffering from respiratory distress comprises a halt in the progression of the respiratory distress (e.g., no deterioration of symptoms) or a delay in the progression of the respiratory distress. The “treatment” may also lead to a partial response (e.g., amelioration of symptoms) or complete response (e.g., disappearance of symptoms) of the subject/patient suffering from respiratory distress. The “treatment” of a subject suffering from respiratory distress may also refer to at least one of the following: obviating the necessity of using artificial respiration, shortening the time duration of artificial respiration, and reducing any adverse effects and/or complication associated with the use of artificial respiration. Each possibility represents a separate embodiment. The treatment of respiratory distress may, inter alia, comprise curative treatment (e.g. disease modifying, preferably leading to a complete response) and palliative treatment (including symptomatic relief). Each possibility represents a separate embodiment.

As used herein, the terms “treatment” and “treating” also include the prevention or prophylactic treatment of respiratory distress, namely the treatment of a subject who does not exhibit signs of a disease or for the purpose of decreasing the risk of developing pathology or further advancement of the early disease. A prophylactic treatment may also refer to preventing recurrence or relapse of a disease or condition in a patient that has previously been afflicted with the disease or condition.

The subject to be treated is a mammal, preferably a human. Among the patient populations for which the compositions and methods of the invention are particularly beneficial, are those afflicted with a non-inflammatory disease or disorder. The diseases and disorders within the scope of the present invention include, but are not limited to, pulmonary hypertension, acute chest syndrome, infectious lung disease, hypoxemia, respiratory failure, respiratory distress syndrome, acute respiratory distress syndrome, acute lung injury, pulmonary embolism, sleep apnea, altitude illness, diabetic ketoacidosis, and respiratory distress caused by lung cancer. Each possibility represents a separate embodiment. In certain embodiments, the disease or disorder includes pulmonary hypertension, acute chest syndrome, hypoxemia, respiratory failure, respiratory distress syndrome, acute respiratory distress syndrome, acute lung injury, pulmonary embolism, sleep apnea, altitude illness, diabetic ketoacidosis, or respiratory distress caused by lung cancer. Each possibility represents a separate embodiment. It is to be understood that treatment of respiratory distress according to the principles of the present invention does not include the treatment of inflammatory diseases and disorders selected from the group consisting of asthma, bronchitis, pleurisy, alveolitis, vasculitis, pneumonia, chronic bronchitis, bronchiectasis, diffuse panbronchiolitis, hypersensitivity pneumonitis, idiopathic pulmonary fibrosis and cystic fibrosis or the treatment of chronic obstructive pulmonary disease (COPD).

In additional aspects and embodiments, treatment may also be given to subjects who are engaged in excessive exercise or heavy smokers thereby being at a risk of experiencing a decline in oxygen saturation levels. Each possibility represents a separate embodiment. In other aspects and embodiments, treatment may be given to hikers traveling at a low oxygen environment at high altitude in order to reduce the risk of development of altitude illness.

A particular patient population to which the compositions and methods of the invention are beneficial relates to individuals who are infected with coronavirus, preferably β-coronavirus, most preferably SARS-COV-2. In this respect, the terms “treatment” and “treating”, used herein interchangeably, refer to the diminishment, alleviation, or amelioration of at least one clinical symptom associated with or caused by coronavirus infection. In one embodiment, the pharmaceutical composition comprising ZEP3, ZEP4, or pharmaceutically acceptable salts thereof is useful in treating COVID-19 patients.

Coronavirus disease 2019 (COVID-19) is an infectious disease caused by SARS-COV-2. Common symptoms of COVID-19 include fever, cough, and shortness of breath. Emergency symptoms include difficulty in breathing, persistent chest pain or pressure, confusion, difficulty in waking, and bluish face or lips which necessitate immediate medical attention. Severe symptoms may include pneumonia, acute respiratory distress syndrome, multi-organ failure and/or death. Currently, there is no known specific treatment for COVID-19 patients. Primary treatment is symptomatic. In severe cases where saturation of peripheral oxygen decreases below approximately 90%, mechanical ventilation is required. The pharmaceutical composition of the present invention exhibits high efficacy in treating and preventing the cytokine storm which often accompanies severe cases of COVID-19. As contemplated herein, the composition of the present invention is highly effective in treating a respiratory distress syndrome including, but not limited to, viral pneumonia, severe acute respiratory syndrome, and acute respiratory distress syndrome (ARDS). Each possibility represents a separate embodiment. In one embodiment, treatment comprises reduction of lung's inflammatory cytokines selected from the group consisting of IL-5, IL-6, and G-CSF. Each possibility represents a separate embodiment. In another embodiment, treatment comprises reduction of lung's inflammatory cytokines selected from the group consisting of IL-1α, IL-6, IL-12, IL-17, KC, MCP-1. TNF-α, CXCL10, and G-CSF. Each possibility represents a separate embodiment. In yet another embodiment, treatment comprises reduction of lung's inflammatory cytokines selected from the group consisting of INF-γ, IL-1α, IL-5, IL-6, IL-12, IL-17, KC, MCP-1, TNF-α, CXCL10, RANTES, G-CSF, and CCL3. Each possibility represents a separate embodiment. In additional embodiments, treatment comprises reduction in the level of T cells, eosinophils, and/or neutrophils in the branchial alveolar fluid. Each possibility represents a separate embodiment. In further embodiments, treatment comprises anti-SARS-COV-2 activity. In other embodiments, treatment comprises inhibition of angiotensin-converting enzyme-2 (ACE-2) mRNA expression.

Determining the therapeutically effective amount is well within the capability of those skilled in the art, especially in light of the disclosure provided herein. The exact formulation, route of administration and dosage can be chosen by the individual physician in view of the patient's condition. The amount of a composition to be administered will be dependent on certain parameters of the subject being treated, for example, weight, age, and the severity of the disease. The pharmaceutical composition may be administered as a single dose or multiple doses in a continuous or intermittent manner. The administration schedule includes once-daily, twice-daily, thrice daily, etc. The term “intermittent” as used herein refers to stopping and starting at either regular or irregular intervals. For example, intermittent administration can be administration every day for a certain period of time or administration in cycles or administration on alternate days. Each possibility represents a separate embodiment.

As used herein, the use of “a” and “an” means “at least one” or “one or more” unless the context clearly dictates otherwise.

As used herein, when a numerical value is preceded by the term “about”, the term “about” is intended to indicate ±10%.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### Example 1

### Efficacy of ZEP3Na in an Acute Inflamed Lung Model

In order to test the efficacy of ZEP3 sodium on lung inflammation, the lipopolysaccharide (LPS)-induced acute lung injury model was used. In particular, LPS was administered intratracheally at doses of 10 or 40 mg/kg body weight (BW) corresponding to 0.2 or 0.8 mg/mouse, respectively.

ZEP3 sodium was administered by inhalation (INH) at doses of 25 or 250 mg/kg BW (0.5 or 5 mg/mouse, respectively), or intratracheally (IT) at doses of 5 or 20 mg/kg BW (0.1 or 0.4 mg/mouse, respectively) 6 hours after LPS administration and its effect was measured 72 hours following administration in the branchial alveolar fluid (BALF) using FACS instrumentation. The results were analyzed by non-parametric statistics and are summarized in Table 1.

TABLE 1

| | LPS 0.8 mg + | LPS 0.8 mg + | LPS 0.2 mg + | LPS 0.2 mg + | LPS 0.8 mg + | LPS 0.8 mg + |
|---|---|---|---|---|---|---|
| | ZEP3Na (INH) | ZEP3Na (INH) | ZEP3Na (IT) | ZEP3Na (IT) | ZEP3Na (IT) | ZEP3Na (IT) |
| | 0.5 mg | 5 mg | 0.1 mg | 0.4 mg | 0.1 mg | 0.4 mg |
| B cells | + | + | ND | ND | ND | ND |
| T cells | − | − | ND | ND | ND | ND |
| Eosinophils | − | − | − | − | ND | ND |
| Neutrophils | − | − | ND | ND | ND | ND |
| Macrophages | + | + | + | + | ND | ND |
| INF-γ | − | − | ND | ND | ND | ND |
| IL-1α | − | − | − | − | ND | ND |
| IL-5 | − | − | ND | ND | ND | ND |
| IL-6 | − | − | − | − | ND | ND |
| IL-10 | + | + | − | − | ND | ND |
| IL-12 | − | − | − | − | − | − |
| IL-17 | − | − | − | − | ND | ND |
| KC | − | − | − | − | ND | ND |
| MCP-1 | − | − | − | − | ND | ND |
| TNF-α | − | − | − | − | − | − |
| CXCL10 | − | − | − | − | ND | ND |
| RANTES | − | − | ND | ND | ND | ND |
| G-CSF | − | − | − | − | ND | ND |
| CCL3 | − | − | ND | ND | ND | ND |

(+) indicates activation; (−) indicates inhibition; (ND) indicates not determined.

The results show that ZEP3Na significantly inhibited the expression and reduced the levels of T cells, eosinophils, neutrophils, INF-γ, IL-1α, IL-5, IL-6, IL-12, IL-17, KC, MCP-1, TNF-α, CXCL10, RANTES, G-CSF, and CCL3 and increased the levels of macrophages and B cells when administered by inhalation following LPS instillation. When administered intratracheally, ZEP3Na significantly inhibited the expression and reduced the levels of eosinophils, IL-1α, IL-6, IL-10, IL-12, IL-17, KC, MCP-1, TNF-α, CXCL10, and G-CSF and increased the levels of macrophages following LPS instillation.

Relative ACE2 mRNA expression in lung tissue was determined as follows: mRNA was extracted from lung tissues, real-time quantitative PCR was employed, and expression of ACE2 gene in relation to rplO gene was measured. The results are summarized in Table 2.

TABLE 2

| Sample 1 | Sample 2 | |
|---|---|---|
| Relative ACE2 mRNA | Relative ACE2 mRNA | |
| expression (Mean ± SEM) | expression (Mean ± SEM) | p-value |
| Saline | LPS 0.8 mg | 0.032 |
| 0.403 ± 0.089 | 1.362 ± 0.157 | |
| | ZEP3 Na 0.1 mg | 0.001 |
| | 1.750 ± 0.320 | |
| | ZEP3 Na 0.4 mg | 0.900 |
| | 0.303 ± 0.025 | |
| | LPS 0.8 mg + ZEP3 Na 0.1 mg | 0.900 |
| | 0.223 ± 0.031 | |
| | LPS 0.8 mg + ZEP3 Na 0.4 mg | 0.900 |
| | 0.805 ± 0.251 | |
| | LPS 0.2 mg | 0.900 |
| | 0.557 ± 0.061 | |
| | LPS 0.2 mg + ZEP3 Na 0.1 mg | 0.900 |
| | 0.672 ± 0.145 | |
| | LPS 0.2 mg + ZEP3 Na 0.4 mg | 0.098 |
| | 1.239 ± 0.132 | |
| LPS 0.8 mg | LPS 0.8 mg + ZEP3 Na 0.1 mg | 0.001 |
| 1.362 ± 0.157 | 0.223 ± 0.031 | |
| | LPS 0.8 mg + ZEP3 Na 0.4 mg | 0.393 |
| | 0.805 ± 0.251 | |
| LPS 0.2 mg | LPS 0.2 mg + ZEP3 Na 0.1 mg | 0.900 |
| 0.557 ± 0.061 | 0.672 ± 0.145 | |
| | LPS 0.2 mg + ZEP3 Na 0.4 mg | 0.105 |
| | 1.239 ± 0.132 | |

While LPS resulted in an increase in ACE2 mRNA expression from a value of 0.4 to a value of 1.36, administration of ZEP3 sodium (0.1 mg/ml) down-regulated ACE2 mRNA expression from 1.36 to a level of 0.22 with a statistical significance of 0.001 indicating efficacy. Without being bound by any theory or mechanism of action, ZEP3Na action is contemplated to be mediated, at least in part, via the down-regulation of ACE2 mRNA.

Overall, the results presented herein demonstrate the efficacy of ZEP3 sodium in the treatment of lung inflammation, including lung inflammation resulting from coronavirus infection. The efficacy is particularly significant when a highly aggressive inflammation is induced using 40 mg/kg LPS. It is contemplated that since ACE2 of the host is used by SARS-COV-2 to enter cells, its reduction by ZEP3Na is indicative for efficacy in the treatment of coronavirus infection.

### Example 2

### Toxicity of ZEP3Na in Intratracheal Administration

The toxicological effect of ZEP3Na administered intratracheally to rats was evaluated. Doses of 0.5, 1.0, and 2.5 mg ZEP3Na dissolved in saline were administered daily into the lungs for a period of 14 days. On day 15, necroscopy and histology of brain, heart, thymus, lungs, spleen, liver, kidneys, and gonads were performed. In addition, blood cell counts and chemistry parameters were counted. Also, the cell distribution (%) of the bronchial alveolar lavage fluid (BALF) was determined by FACS instrumentation for B cells, T cells, neutrophils, and macrophages.

No mortality or abnormal signs of behavior were observed. The body weight of the rats remained similar to that of the control group (saline). Necroscopy and histology of the inspected organs revealed no pathological changes in relation to the tested item. Furthermore, blood counts and chemistry were comparable to the reference items. No changes were found in the distribution of B cells, T cells, neutrophils, and macrophages. Taken together, these results show that intratracheal (IT) administration of ZEP3Na at concentrations of up to 2.5 mg daily for 14 days did not result in any detectable adverse effects in all parameters that were tested.

### Example 3

### Efficacy of ZEP3Na in Treating Acute Respiratory Distress Syndrome

The effect of ZEP3Na administered intratracheally in a mice model of acute respiratory distress syndrome was evaluated. BALB/c mice were anaesthetized and orally intubated with a sterile plastic catheter and challenged with intratracheal instillation of 0.2 mg or 0.8 mg of LPS dissolved in 50 μL of PBS. Naïve mice (without LPS instillation) were injected with the same volume of saline to serve as controls. Six hours following LPS instillation, mice were administered intratracheally with ZEP3Na at two doses, namely 0.1 and 0.4 mg. The study was terminated 72 hours after LPS challenge to collect tissues for analysis.

Animal body weights were determined daily during the study. No major changes in body weight were noted. BALF samples were processed for differential cell count by FACS to determine the cellular composition of B cells, T cells, eosinophils, neutrophils, and macrophages/dendritic cells. The results are summarized in Table 3.

TABLE 3

| | | | | | | Macrophages |
|---|---|---|---|---|---|---|
| | | B cells | T cells | Eosinophils | Neutrophils | & dendritic |
| Group | # | (%) | (%) | (%) | (%) | cells (%) |
| 1-LPS | 11 | 0.029 | 0.417 | 0.451 | 93.477 | 0.323 |
| 0.8 mg | 12 | 0.033 | 0.452 | 0.083 | 92.314 | 0.677 |
| | 13 | 0.030 | 0.557 | 0.849 | 92.441 | 0.534 |
| | 15 | 0.014 | 0.451 | 0.421 | 93.691 | 0.173 |
| | 16 | 0.056 | 1.019 | 1.415 | 85.360 | 1.467 |
| | 17 | 0.041 | 0.760 | 0.731 | 91.720 | 0.928 |
| | 18 | 0.028 | 0.609 | 0.398 | 93.173 | 0.662 |
| | 19 | 0.017 | 0.398 | 0.586 | 92.935 | 0.437 |
| | 20 | 0.024 | 0.462 | 0.418 | 93.998 | 0.103 |
| Mean | | 0.030 | 0.569 | 0.595 | 92.123 | 0.589 |
| SEM | | 0.004 | 0.068 | 0.126 | 0.879 | 0.140 |
| 2-ZEP3Na | 21 | 0.320 | 3.447 | 0.192 | 8.302 | 69.267 |
| 0.1 mg | 22 | 0.208 | 3.524 | 0.556 | 7.056 | 65.626 |
| | 23 | 0.091 | 4.627 | 0.185 | 4.882 | 66.568 |
| | 24 | 0.218 | 2.747 | 0.196 | 7.770 | 65.527 |
| | 25 | 0.150 | 2.636 | 0.692 | 5.279 | 65.957 |
| | 26 | 0.089 | 1.788 | 0.058 | 3.207 | 70.380 |
| | 27 | 0.103 | 2.856 | 0.261 | 3.871 | 68.287 |
| | 28 | 0.162 | 2.681 | 0.828 | 12.158 | 64.068 |
| | 29 | 0.147 | 3.118 | 0.606 | 8.592 | 65.131 |
| | 30 | 0.138 | 3.354 | 0.546 | 7.044 | 66.345 |
| Mean | | 0.163 | 3.078 | 0.412 | 6.816 | 66.715 |
| SEM | | 0.022 | 0.235 | 0.083 | 0.835 | 0.626 |
| 3-ZEP3Na | 31 | 0.310 | 2.519 | 0.151 | 3.325 | 72.760 |
| 0.4 mg | 33 | 0.225 | 3.322 | 0.400 | 7.965 | 67.646 |
| | 34 | 0.162 | 1.247 | 0.136 | 2.600 | 73.862 |
| | 35 | 0.154 | 1.675 | 0.176 | 3.496 | 72.372 |
| | 36 | 0.106 | 1.513 | 0.138 | 2.405 | 77.634 |
| | 37 | 0.100 | 1.348 | 0.390 | 3.740 | 73.286 |
| | 38 | 0.160 | 1.817 | 0.192 | 3.111 | 72.368 |
| | 39 | 0.223 | 2.755 | 0.540 | 7.438 | 67.355 |
| | 40 | 0.144 | 1.654 | 0.366 | 4.152 | 72.688 |
| Mean | | 0.176 | 1.983 | 0.277 | 4.248 | 72.219 |
| SEM | | 0.022 | 0.238 | 0.050 | 0.678 | 1.043 |
| 4-LPS | 41 | 0.118 | 0.991 | 0.170 | 6.007 | 71.986 |
| 0.8 mg + | 42 | 0.102 | 1.527 | 0.064 | 2.454 | 72.475 |
| ZEP3Na | 43 | 0.048 | 0.804 | 1.024 | 77.259 | 1.316 |
| 0.1 mg | 44 | 0.030 | 0.573 | 0.755 | 86.115 | 0.956 |
| | 45 | 0.045 | 0.865 | 0.646 | 82.630 | 1.424 |
| | 46 | 0.489 | 5.340 | 3.005 | 57.391 | 16.916 |
| | 47 | 0.052 | 0.937 | 0.568 | 82.523 | 1.107 |
| | 48 | 0.572 | 8.033 | 0.840 | 54.903 | 14.732 |
| | 49 | 0.017 | 0.675 | 0.425 | 82.147 | 0.713 |
| Mean | | 0.164 | 2.194 | 0.833 | 59.048 | 20.180 |
| SEM | | 0.070 | 0.883 | 0.290 | 11.018 | 10.055 |
| 5-LPS | 52 | 0.014 | 0.383 | 0.303 | 77.586 | 0.470 |
| 0.8 mg + | 53 | 0.011 | 0.495 | 0.448 | 88.700 | 0.297 |
| ZEP3Na | 54 | 0.028 | 0.512 | 0.323 | 84.629 | 0.271 |
| 0.4 mg | 55 | 0.024 | 0.577 | 0.298 | 80.400 | 0.442 |
| | 57 | 0.167 | 1.800 | 1.427 | 39.818 | 30.597 |
| | 58 | 0.138 | 2.098 | 0.195 | 3.154 | 68.407 |
| | 59 | 0.026 | 0.609 | 0.530 | 84.929 | 0.798 |
| | 60 | 0.022 | 0.685 | 0.280 | 80.922 | 0.764 |
| Mean | | 0.054 | 0.895 | 0.475 | 67.517 | 12.756 |
| SEM | | 0.022 | 0.234 | 0.141 | 10.692 | 8.779 |
| 6-Saline | 61 | 0.129 | 6.078 | 0.129 | 12.220 | 66.142 |
| | 62 | 0.115 | 4.410 | 0.000 | 19.112 | 61.257 |
| | 63 | 0.119 | 3.482 | 0.060 | 31.607 | 53.482 |
| | 64 | 0.075 | 1.620 | 0.017 | 2.149 | 73.022 |
| | 65 | 0.120 | 1.496 | 0.026 | 3.198 | 68.290 |
| Mean | | 0.112 | 3.417 | 0.046 | 13.657 | 64.439 |
| SEM | | 0.010 | 0.866 | 0.023 | 5.458 | 3.329 |
| 7-LPS | 71 | 0.057 | 1.107 | 0.894 | 84.469 | 0.525 |
| 0.2 mg | 72 | 0.018 | 0.384 | 0.427 | 89.975 | 0.600 |
| | 73 | 0.061 | 0.886 | 0.904 | 89.817 | 1.483 |
| | 74 | 0.050 | 1.001 | 1.217 | 87.334 | 2.687 |
| | 75 | 0.045 | 0.916 | 0.816 | 89.344 | 1.045 |
| | 76 | 0.040 | 0.748 | 0.818 | 89.837 | 1.626 |
| | 77 | 0.026 | 0.699 | 0.275 | 91.067 | 0.914 |
| | 78 | 0.042 | 0.559 | 0.194 | 87.831 | 0.811 |
| | 79 | 0.075 | 1.469 | 0.523 | 89.574 | 1.963 |
| | 80 | 0.080 | 1.464 | 0.712 | 87.335 | 2.122 |
| Mean | | 0.049 | 0.923 | 0.678 | 88.658 | 1.378 |
| SEM | | 0.006 | 0.112 | 0.101 | 0.608 | 0.228 |
| 8-LPS | 81 | 0.040 | 0.940 | 0.247 | 84.112 | 2.218 |
| 0.2 mg + | 82 | 0.064 | 0.742 | 0.204 | 84.283 | 1.674 |
| ZEP3Na | 83 | 0.259 | 5.066 | 0.223 | 69.683 | 5.674 |
| 0.1 mg | 84 | 0.230 | 3.187 | 0.251 | 74.854 | 2.257 |
| | 85 | 0.080 | 1.024 | 0.143 | 81.436 | 0.618 |
| | 86 | 0.054 | 1.045 | 0.176 | 79.270 | 1.800 |
| | 87 | 0.069 | 1.327 | 0.180 | 71.074 | 1.175 |
| | 88 | 0.053 | 0.892 | 0.265 | 80.725 | 5.084 |
| | 89 | 0.045 | 1.131 | 0.225 | 85.962 | 1.252 |
| | 90 | 0.075 | 1.271 | 0.192 | 82.883 | 1.516 |
| Mean | | 0.097 | 1.662 | 0.210 | 79.428 | 2.327 |
| SEM | | 0.025 | 0.437 | 0.012 | 1.800 | 0.533 |
| 9-LPS | 91 | 0.054 | 0.833 | 0.159 | 85.551 | 1.327 |
| 0.2 mg + | 92 | 0.029 | 0.582 | 0.151 | 87.483 | 0.529 |
| ZEP3Na | 93 | 0.019 | 0.685 | 0.192 | 77.212 | 0.437 |
| 0.4 mg | 94 | 0.100 | 1.665 | 0.238 | 72.881 | 1.661 |
| | 95 | 0.153 | 2.779 | 0.345 | 9.005 | 60.592 |
| | 96 | 0.286 | 0.977 | 0.577 | 3.108 | 75.765 |
| | 98 | 0.103 | 1.174 | 0.237 | 3.716 | 73.903 |
| | 99 | 0.037 | 0.843 | 0.288 | 81.207 | 1.626 |
| | 100 | 0.045 | 1.035 | 0.243 | 77.353 | 4.399 |
| Mean | | 0.092 | 1.175 | 0.270 | 55.279 | 24.471 |
| SEM | | 0.028 | 0.226 | 0.043 | 12.597 | 11.493 |

Intratracheal LPS administration induced severe lung inflammation that was reflected by altered cell composition in LPS treated animals compared to animals that were not treated with LPS. A higher percentage of neutrophils and eosinophils, and a lower percentage of macrophages/dendritic cells in BALF of LPS-treated animals as compared to the control group that received saline was observed.

In animals that were challenged with 0.2 mg LPS, ZEP3Na treatment resulted in a significant decrease in bronchoalveolar eosinophils percentage as compared to untreated mice that received 0.2 mg LPS (Table 3).

LPS at 0.2 and 0.8 mg per mouse, elicited an increase of eosinophils and neutrophils and a decrease in the presence of macrophages. In 0.2 mg LPS-inflamed mice, ZEP3Na at 0.1 and 0.4 mg significantly inhibited the level of eosinophils. These cells along with neutrophils, have a central role in the immune response to various pathogens. Their accumulation within the lung is a leading cause for acute lung injury (ALI) and acute respiratory distress syndrome (ARDS) disorders. In addition, elevation in the presence of macrophages by ZEP3Na is contemplated as pivotal in restoring proper lung function that has been compromised by infection.

To detect cytokine release in lungs fluid, cytokine concentrations were assessed in BALF samples using high sensitivity multiplex ELISA assays for: IFN-γ, IL-1α, IL-5, IL-6, IL-10, IL-12 (p70), IL-17A, KC, MCP-1, TNF-α, IP-10, RANTES, G-CSF, MIP-1α and RANTES. ZEP3Na administered intratracheally at 0.1 and 0.4 mg per mouse after instillation of 0.2 mg LPS inhibited the concentration of following interleukins: IL-1α, IL-6, IL-10, IL-12, IL-17, KC, MCP-1, TNF-α and G-CSF. For 0.8 mg LPS treated mice, ZEP-3Na 0.1 and 0.4 mg significantly inhibited the production of IL-12 and TNF-α. The results of IL-12, IL-17, KC, MCP-1, and G-CSF are summarized in Table 4.

TABLE 4

| | | IL-12 (p70) | IL-17A | KC | MCP-1 | G-CSF |
|---|---|---|---|---|---|---|
| Group | # | (pg/ml) | (pg/ml) | (pg/ml) | (pg/ml) | (pg/ml) |
| 1-LPS | 11 | 153.16 | 66.46 | 949.85 | 134.29 | 322.60 |
| 0.8 mg | 12 | 13.70 | 2.18 | 69.72 | 18.12 | 5.06 |
| | 13 | 24.95 | 32.64 | 626.12 | 105.01 | 268.77 |
| | 15 | 14.85 | 12.60 | 686.25 | 1085.74 | 1235.76 |
| | 16 | 10.92 | 3.26 | 605.93 | 107.96 | 163.91 |
| | 17 | 298.18 | 246.57 | 347.76 | 107.15 | 417.03 |
| | 18 | 399.36 | 561.13 | 1087.66 | 95.00 | 290.54 |
| | 19 | 12.09 | 4.16 | 151.00 | undetected | 1046.89 |
| | 20 | 15.58 | 3.08 | 391.13 | 1424.86 | 1543.76 |
| Mean | | 104.75 | 103.56 | 546.16 | 342.02 | 588.26 |
| SEM | | 49.25 | 62.90 | 113.79 | 186.16 | 180.79 |
| 2-ZEP3Na | 21 | 9.22 | undetected | 46.08 | undetected | 2.37 |
| 0.1 mg | 22 | 9.10 | undetected | 58.58 | undetected | 2.37 |
| | 23 | 8.79 | undetected | 58.91 | undetected | 1.99 |
| | 24 | 7.39 | 0.63 | 89.58 | undetected | 5.06 |
| | 25 | 5.38 | undetected | 95.87 | undetected | 2.76 |
| | 26 | 4.32 | undetected | 47.76 | undetected | undetected |
| | 27 | undetected | undetected | 43.47 | undetected | 3.53 |
| | 28 | 10.92 | 2.12 | 64.27 | undetected | 1.99 |
| | 29 | 8.79 | 0.73 | 42.44 | undetected | 2.76 |
| | 30 | 9.22 | undetected | 45.89 | undetected | 3.15 |
| Mean | | 7.31 | 0.35 | 59.28 | 0.00 | 2.60 |
| SEM | | 1.08 | 0.40 | 6.06 | NA | 0.43 |
| 3-ZEP3Na | 31 | 8.49 | 2.02 | 56.01 | 11.53 | 7.65 |
| 0.4 mg | 33 | 7.27 | 1.13 | 58.30 | undetected | 3.47 |
| | 34 | 7.92 | 1.36 | 63.33 | 11.29 | 2.20 |
| | 35 | 8.49 | 1.47 | 62.30 | undetected | 6.40 |
| | 36 | 5.27 | 1.20 | 71.12 | 9.93 | undetected |
| | 37 | 9.45 | 1.06 | 68.10 | undetected | 2.20 |
| | 38 | 7.21 | 1.20 | 52.39 | undetected | undetected |
| | 39 | 5.22 | 0.73 | 46.36 | undetected | 3.47 |
| | 40 | 8.54 | 1.14 | 160.65 | 11.29 | 3.89 |
| Mean | | 7.54 | 1.26 | 70.95 | 4.89 | 3.26 |
| SEM | | 0.49 | 0.12 | 11.50 | 2.91 | 0.97 |
| 4-LPS | 41 | 6.33 | undetected | 39.18 | undetected | undetected |
| 0.8 mg + | 42 | 5.38 | undetected | 29.08 | undetected | undetected |
| ZEP3Na | 43 | 235.43 | 1287.47 | 847.35 | 70.21 | 250.42 |
| 0.1 mg | 44 | 103.27 | 55.37 | 522.41 | 76.09 | 141.30 |
| | 45 | 192.86 | 719.90 | 798.01 | 93.90 | 164.43 |
| | 46 | 41.94 | 51.28 | 253.77 | 58.01 | 89.05 |
| | 47 | 414.83 | 5285.06 | 831.88 | 114.01 | 416.35 |
| | 48 | 27.99 | 77.04 | 234.34 | 53.91 | 50.11 |
| | 49 | 88.80 | 65.83 | 638.49 | 165.51 | 131.80 |
| Mean | | 124.09 | 837.99 | 466.06 | 70.18 | 138.16 |
| SEM | | 45.23 | 651.97 | 111.45 | 19.76 | 49.83 |
| 5-LPS | 52 | 7.58 | 7.23 | 1115.74 | 810.24 | 491.06 |
| 0.8 mg + | 53 | 215.64 | 265.69 | 1099.42 | 166.84 | 209.62 |
| ZEP3Na | 54 | 368.44 | 1207.05 | 1195.30 | 79.29 | 268.52 |
| 0.4 mg | 55 | 12.63 | 13.05 | 488.61 | 307.60 | 300.90 |
| | 57 | 7.60 | 2.04 | 54.20 | 15.45 | 7.65 |
| | 58 | undetected | 0.52 | 57.06 | undetected | 3.47 |
| | 59 | 194.02 | 136.95 | 981.92 | 83.52 | 124.37 |
| | 60 | 14.83 | 6.24 | 1193.59 | 62.54 | 116.99 |
| Mean | | 102.59 | 204.85 | 773.23 | 190.69 | 190.32 |
| SEM | | 52.72 | 147.05 | 175.92 | 101.57 | 57.81 |
| 6-Saline | 61 | undetected | undetected | 45.46 | undetected | 1.99 |
| | 62 | undetected | 1.50 | 34.54 | undetected | undetected |
| | 63 | undetected | 1.18 | 30.89 | undetected | 1.99 |
| | 64 | undetected | undetected | 20.27 | undetected | undetected |
| | 65 | undetected | 0.63 | 35.62 | undetected | undetected |
| Mean | | 0.00 | 0.66 | 33.36 | 0.00 | 0.79 |
| SEM | | NA | 0.39 | 4.06 | NA | 0.77 |
| 7-LPS | 71 | 500.42 | 1787.92 | 1013.50 | 92.66 | 349.50 |
| 0.2 mg | 72 | 14.82 | 7.84 | 767.87 | 456.42 | 445.12 |
| | 73 | 125.09 | 65.82 | 701.19 | 144.80 | 227.25 |
| | 74 | 204.01 | 110.19 | 829.85 | 127.10 | 264.32 |
| | 75 | 34.42 | 30.83 | 741.15 | 83.08 | 152.26 |
| | 76 | 37.20 | 44.44 | 1041.29 | 154.77 | 130.58 |
| | 77 | 24.79 | 23.32 | 1099.54 | 160.76 | 264.70 |
| | 78 | 9.75 | 24.32 | 298.66 | 141.51 | 335.46 |
| | 79 | 270.46 | 384.99 | 490.83 | 182.18 | 263.94 |
| | 80 | 417.87 | 3123.70 | 670.42 | 164.26 | 340.78 |
| Mean | | 163.88 | 560.34 | 765.43 | 170.75 | 277.39 |
| SEM | | 179.62 | 1053.21 | 249.54 | 105.07 | 94.84 |
| 8-LPS | 81 | 247.16 | 553.70 | 782.52 | 107.46 | 175.22 |
| 0.2 mg + | 82 | 386.37 | 1615.14 | 1528.47 | 158.19 | 144.90 |
| ZEP3Na | 83 | 66.34 | 285.28 | 503.00 | 142.05 | 109.99 |
| 0.1 mg | 84 | 199.27 | 1776.16 | 672.71 | 85.01 | 383.77 |
| | 85 | 322.18 | 862.84 | 1548.79 | 91.65 | 239.49 |
| | 86 | 422.04 | 989.92 | 1057.28 | 135.00 | 199.64 |
| | 87 | 317.94 | 458.49 | 1346.32 | 216.90 | 249.05 |
| | 88 | 97.45 | 111.01 | 808.21 | 114.81 | 93.60 |
| | 89 | 202.28 | 281.41 | 1086.03 | 109.95 | 177.33 |
| | 90 | 388.69 | 308.73 | 903.77 | 171.38 | 106.48 |
| Mean | | 264.97 | 724.27 | 1023.71 | 133.24 | 187.95 |
| SEM | | 39.00 | 183.40 | 113.18 | 12.81 | 27.61 |
| 9-LPS | 91 | 408.22 | 386.05 | 1154.03 | 240.17 | 176.85 |
| 0.2 mg + | 92 | 471.74 | 1361.97 | 1559.44 | 115.81 | 200.57 |
| ZEP3Na | 93 | 15.41 | 15.26 | 1143.16 | 823.17 | 585.70 |
| 0.4 mg | 94 | 262.69 | 3386.75 | 1153.90 | 151.55 | 211.19 |
| | 95 | 15.41 | 22.20 | 127.59 | 14.15 | 8.45 |
| | 96 | 11.53 | 4.57 | 63.82 | undetected | undetected |
| | 98 | 8.09 | 2.99 | 75.31 | undetected | undetected |
| | 99 | 115.45 | 21.13 | 656.41 | 102.63 | 138.72 |
| | 100 | 140.60 | 55.67 | 826.24 | 174.95 | 227.63 |
| Mean | | 161.01 | 584.07 | 751.10 | 180.27 | 172.12 |
| SEM | | 59.93 | 380.21 | 185.00 | 96.41 | 68.61 |

It is contemplated that the inhibition in the production of IL-12, IL-17, and MCP-1 by ZEP3Na indicates the efficacy of ZEP3Na in the treatment of pulmonary embolism, obstructive sleep apnea syndrome, and altitude illness. These results further indicate that ZEP3Na is effective in treating respiratory distress, including ARDS caused by coronavirus infection.

### Example 4

### Oxygen Saturation

Evaluation of the effect of ZEP3Na administration via inhalation on oxygen saturation in a mouse model of inflamed lungs by the lipopolysaccharides (LPS) was performed. Oxygen saturation testing was performed at several time points on mice that have been anesthetized by isoflurane. A sensor was attached to the hind foot of the mice and the percentage of oxygen in the blood was measured using the Rodent Pulse Oximeter instrument (Kent Scientific).

In particular, Balb/C female mice (7-9 weeks old, 18-21 gram, Envigo, Israel; 10 mice in each group) were anaesthetized and orally intubated with a sterile plastic catheter for intratracheal instillation of 800 μg of LPS dissolved in 50 μL of PBS. Following LPS intratracheal instillation, a marked reduction in blood oxygen saturation levels was detected. ZEP3Na was then administered by inhalation at two doses 6 h and 14 h after LPS administration and the effect of ZEP3Na on blood oxygen saturation was monitored 0, 2, 6, 12, 24 and 48 hours following ZEP3Na administration. The results are summarized in Table 5. Saturation data was analyzed by ANOVA followed by Tukey-Kramer test. Significance level was defined as ∝=0.05. Animal weight was shown to be substantially constant throughout the experiment.

TABLE 5

| Group | Mouse # | 0 h | 2 h | 6 h | 12 h | 24 h | 48 h |
|---|---|---|---|---|---|---|---|
| 1. LPS | 9845 | 96 | 72 | 83 | 82 | 84 | 88 |
| | 9846 | 94 | 64 | 83 | 77 | 86 | 81 |
| | 9848 | 97 | 72 | 82 | 85 | 82 | 90 |
| | 9849 | 98 | 58 | 85 | 72 | 90 | 87 |
| | 9852 | 96 | 73 | 85 | 70 | 88 | 92 |
| | 9853 | 97 | 77 | 86 | 84 | 93 | 90 |
| | 9854 | 99 | 68 | 84 | 81 | 90 | 89 |
| | 9855 | 94 | 64 | 87 | 82 | 91 | 88 |
| | 9858 | 99 | 64 | 82 | 76 | 87 | 80 |
| | 9859 | 97 | 72 | 84 | 80 | 84 | 88 |
| | Mean | 96.7 | 68.4 | 84.1 | 78.9 | 87.5 | 87.3 |
| | SEM | 0.53 | 1.73 | 0.50 | 1.51 | 1.06 | 1.16 |
| 2. LPS + | 9847 | 99 | 77 | 81 | 92 | 86 | 91 |
| ZEP3Na-10 | 9850 | 93 | 67 | 88 | 96 | 87 | 91 |
| mg 6 h | 9851 | 97 | 78 | 88 | 95 | 89 | 94 |
| | 9856 | 98 | 61 | 87 | 93 | 88 | 97 |
| | 9857 | 99 | 78 | 87 | 92 | 90 | 97 |
| | 9860 | 99 | 71 | 91 | 90 | 86 | 91 |
| | 9861 | 95 | 59 | 82 | 94 | 83 | 97 |
| | 9862 | 99 | 72 | 84 | 95 | 90 | 91 |
| | 9863 | 98 | 74 | 85 | 94 | 92 | 89 |
| | 9864 | 95 | 63 | 89 | 94 | 99 | 91 |
| | Mean | 97.2 | 70.0 | 86.2 | 93.5 | 89.0 | 92.9 |
| | SEM | 0.64 | 2.14 | 0.95 | 0.53 | 1.30 | 0.92 |
| 3. LPS + | 9865 | 97 | 70 | 86 | 92 | 96 | 91 |
| ZEP3Na-10 mg | 9866 | 96 | 68 | 88 | 90 | 94 | 91 |
| 6 h + 14 h | 9867 | 99 | 71 | 89 | 94 | 88 | 92 |
| | 9868 | 94 | 80 | 89 | 97 | 86 | 92 |
| | 9869 | 99 | 73 | 86 | 92 | 93 | 97 |
| | 9870 | 94 | 64 | 92 | 95 | 92 | 97 |
| | 9871 | 93 | 75 | 81 | 88 | 93 | 91 |
| | 9872 | 97 | 62 | 82 | 90 | 96 | 94 |
| | 9873 | 95 | 71 | 90 | 91 | 90 | 94 |
| | 9874 | 98 | 77 | 88 | 96 | 93 | 91 |
| | Mean | 96.2 | 71.1 | 87.1 | 92.5 | 92.1 | 93.0 |
| | SEM | 0.64 | 1.66 | 1.03 | 0.87 | 0.97 | 0.72 |
| 4. LPS + | 9890 | 95 | 69 | 86 | 93 | 93 | 93 |
| ZEP3Na-50 | 9891 | 97 | 81 | 90 | 89 | 94 | 95 |
| mg 6 h | 9892 | 99 | 72 | 88 | 94 | 95 | 98 |
| | 9893 | 99 | 73 | 86 | 95 | 90 | 98 |
| | 9894 | 99 | 79 | 87 | 96 | 95 | 98 |
| | 9895 | 95 | 65 | 88 | 96 | 99 | 98 |
| | 9896 | 99 | 66 | 85 | 94 | 93 | 99 |
| | 9897 | 97 | 72 | 86 | 96 | 92 | 97 |
| | 9898 | 95 | 71 | 84 | 94 | 92 | 96 |
| | 9899 | 97 | 79 | 87 | 98 | 97 | 98 |
| | Mean | 97.2 | 72.7 | 86.7 | 94.5 | 94.0 | 97.0 |
| | SEM | 0.53 | 1.64 | 0.51 | 0.72 | 0.79 | 0.55 |
| 5. LPS + | 9900 | 97 | 63 | 88 | 92 | 90 | 99 |
| ZEP3Na-50 mg | 9901 | 94 | 71 | 90 | 94 | 91 | 97 |
| 6 h + 14 h | 9902 | 96 | 69 | 84 | 90 | 93 | 99 |
| | 9903 | 97 | 92 | 89 | 94 | 98 | 97 |
| | 9904 | 99 | 73 | 86 | 93 | 99 | 91 |
| | 9905 | 98 | 75 | 86 | 95 | 98 | 98 |
| | 9906 | 99 | 72 | 88 | 95 | 91 | 99 |
| | 9907 | 95 | 76 | 85 | 96 | 94 | 98 |
| | 9908 | 94 | 66 | 85 | 97 | 93 | 96 |
| | 9909 | 97 | 61 | 87 | 94 | 91 | 97 |
| | Mean | 96.6 | 71.8 | 86.8 | 94.0 | 93.8 | 97.1 |
| | SEM | 0.55 | 2.60 | 0.58 | 0.60 | 1.01 | 0.71 |
| 6. Saline | 9875 | 97 | 93 | 97 | 98 | 96 | 97 |
| | 9876 | 98 | 89 | 94 | 96 | 98 | 99 |
| | 9877 | 95 | 95 | 96 | 96 | 98 | 97 |
| | 9878 | 98 | 92 | 97 | 95 | 98 | 98 |
| | 9879 | 99 | 90 | 98 | 96 | 99 | 98 |
| | 9880 | 95 | 94 | 95 | 98 | 98 | 99 |
| | 9881 | 96 | 94 | 98 | 98 | 99 | 97 |
| | 9882 | 98 | 92 | 96 | 95 | 99 | 97 |
| | 9883 | 99 | 90 | 98 | 96 | 98 | 98 |
| | 9884 | 97 | 93 | 97 | 96 | 98 | 99 |
| | Mean | 97.2 | 92.2 | 96.6 | 96.4 | 98.1 | 97.9 |
| | SEM | 0.44 | 0.60 | 0.40 | 0.35 | 0.26 | 0.26 |

The results show that administration of ZEP3Na by inhalation significantly increased oxygen saturation levels that have been reduced by LPS. The results are statistically significant 12 hours following ZEP3Na treatment with up to approximately 20% elevation in oxygen saturation levels as compared to LPS with no treatment.

It will be appreciated by persons skilled in the art that the present invention is not limited by what has been particularly shown and described herein above. Rather, the scope of the invention is defined by the claims that follow.

1.-47. (canceled)

48. A method of treating or preventing respiratory distress in a subject in need thereof, the method comprising administering to the subject a pharmaceutical composition comprising a therapeutically effective amount of a peptide selected from pGlu-Asn-Trp-Lys(Octanoyl)-OH (SEQ ID NO: 1), pGlu-Asn-Trp-Thr-OH (SEQ ID NO: 2), and pharmaceutically acceptable salts thereof.

49. The method according to claim 48, wherein treating respiratory distress comprises increasing a decreased level of blood oxygen saturation in the subject or decreasing an increased level of respiratory rate of the subject.

50. The method according to claim 48, wherein the subject is afflicted with a disease or disorder selected from pulmonary hypertension, acute chest syndrome, infectious lung disease, hypoxemia, respiratory failure, respiratory distress syndrome, acute respiratory distress syndrome, acute lung injury, pulmonary embolism, sleep apnea, altitude illness, diabetic ketoacidosis, and respiratory distress caused by lung cancer; or wherein the subject is afflicted with a disease or disorder selected from pulmonary hypertension, acute chest syndrome, hypoxemia, respiratory failure, respiratory distress syndrome, acute respiratory distress syndrome, acute lung injury, pulmonary embolism, sleep apnea, altitude illness, diabetic ketoacidosis, and respiratory distress caused by lung cancer; or wherein the subject is engaged in excessive exercise or practices excess smoking.

51. The method according to claim 48, wherein the peptide has an amino acid sequence as set forth in SEQ ID NO: 1 or a salt thereof.

52. The method according to claim 51, wherein the peptide is the sodium salt of the peptide having an amino acid sequence as set forth in SEQ ID NO: 1.

53. The method according to claim 48, wherein the peptide has an amino acid sequence as set forth in SEQ ID NO: 2 or a salt thereof.

54. The method according to claim 53, wherein the peptide is the sodium salt of the peptide having an amino acid sequence as set forth in SEQ ID NO: 2.

55. The method according to claim 48, wherein the pharmaceutical composition is formulated for administration via inhalation; or wherein the pharmaceutical composition is formulated for intratracheal administration; or wherein the pharmaceutical composition is formulated for intrabronchial administration; or wherein the pharmaceutical composition is formulated for intranasal administration; or wherein the pharmaceutical composition is formulated for intra-alveolar administration; or wherein the pharmaceutical composition is formulated in a form of a solution, a suspension, a powder, a spray or an aerosol.

56. The method according to claim 48, wherein the pharmaceutical composition further comprises a pharmaceutically acceptable excipient comprising at least one of a binder, a filler, a diluent, a surfactant or emulsifier, a glidant or lubricant, a buffering or pH adjusting agent, a tonicity enhancing agent, a wetting agent, a thickening agent, a suspending agent, a preservative, an antioxidant, a solvent, a flavoring agent, a colorant, and a mixture or combination thereof.

57. A method of treating a subject infected with coronavirus, the method comprising the step of administering to the subject a pharmaceutical composition comprising a peptide of pGlu-Asn-Trp-Lys(Octanoyl)-OH (SEQ ID NO: 1), pGlu-Asn-Trp-Thr-OH (SEQ ID NO: 2), or a pharmaceutically acceptable salt of SEQ ID NO: 1 or SEQ ID NO: 2.

58. The method according to claim 57, wherein the coronavirus is β-coronavirus.

59. The method according to claim 58, wherein the β-coronavirus is Severe Acute Respiratory Syndrome (SARS)-COV-2.

60. The method according to claim 57, wherein the subject suffers from a cytokine storm; or wherein the subject suffers from a respiratory syndrome.

61. The method according to claim 60, wherein the respiratory syndrome is selected from the group consisting of viral pneumonia, lung angioedema, pulmonary embolism, severe acute respiratory syndrome, and acute respiratory distress syndrome (ARDS).

62. The method according to claim 57, wherein the peptide has an amino acid sequence as set forth in SEQ ID NO: 1 or a salt thereof; or wherein the peptide is the sodium salt of the peptide having an amino acid sequence as set forth in SEQ ID NO: 1.

63. The method according to claim 57, wherein the peptide has an amino acid sequence as set forth in SEQ ID NO: 2 or a salt thereof; or wherein the peptide is the sodium salt of the peptide having an amino acid sequence as set forth in SEQ ID NO: 2.

64. The method according to claim 57, wherein the pharmaceutical composition is formulated for administration via inhalation; or wherein the pharmaceutical composition is formulated for intratracheal administration; or wherein the pharmaceutical composition is formulated for intrabronchial administration; or wherein the pharmaceutical composition is formulated for intranasal administration; or wherein the pharmaceutical composition is formulated for intra-alveolar administration; or wherein the pharmaceutical composition is formulated in a form of a solution, a suspension, a powder, a spray or an aerosol.

65. The method according to claim 57, wherein the pharmaceutical composition further comprises a pharmaceutically acceptable excipient comprising at least one of a binder, a filler, a diluent, a surfactant or emulsifier, a glidant or lubricant, a buffering or pH adjusting agent, a tonicity enhancing agent, a wetting agent, a thickening agent, a suspending agent, a preservative, an antioxidant, a solvent, a flavoring agent, a colorant, and a mixture or combination thereof.

66. The method according to claim 57, wherein the pharmaceutical composition is co-administered with at least one other active agent.

67. The method according to claim 66, wherein the at least one other active agent is an anti-viral agent; or wherein the at least one other active agent is selected from the group consisting of chloroquine, quercetin, vitamin D, hispidulin, cirsimaritin, sulfasalazine, artemisin, curcuma, and a mixture or combination thereof; or wherein co-administration is performed in a regimen selected from administration of a single combined composition, separate individual compositions administered substantially at the same time, and separate individual compositions administered under separate schedules.
